SEQUENCE LISTING (6 sequences)

SEQ ID NO: 1 - DNA, 26 bp, Homo sapiens
gctgtccaatttctattaaaggttcc

SEQ ID NO: 2 - DNA, 20 bp, Homo sapiens
ggcagaatccagatgctcaa

SEQ ID NO: 3 - DNA, 22 bp, Homo sapiens
gagactctggcatgctaactag

SEQ ID NO: 4 - DNA, 21 bp, Homo sapiens
ggacatctaagggcatcacag

SEQ ID NO: 5 - DNA, 29 bp, Homo sapiens
tgttccctaagtccaactactaaactggg

SEQ ID NO: 6 - DNA, 22 bp, Homo sapiens
tgctcaatctcgggtggctgaa

(19) United States
(12) Patent Application Publication
(10) Pub. No.: US 2019/0177798 A1
(43) Pub. Date: Jun. 13, 2019

(54) BIOMARKER FOR PRENATAL DIAGNOSIS OF TWIN-TO-TWIN TRANSFUSION SYNDROME

(71) Applicants: UNIVERSIDADE DO PORTO, Porto (PT); IPATIMUP (INSTITUTO DE PATOLOGIA E IMUNOLOGIA MOLECULAR DA UNIVERSIDADE DO PORTO), Porto (PT)

(72) Inventors: Berta Cecilia CAMPOS LIMA DE CARVALHO, Porto (PT); Filipa ABREU GOMES DE CARVALHO, Porto (PT); Carla Isabel GONCALVES DE OLIVEIRA, Porto (PT); Patricia Joana MORAIS FERREIRA OLIVEIRA, Porto (PT); Alexandra MATIAS PEREIRA DA CUNHA COELHO DE MACEDO, Porto (PT)

(21) Appl. No.: 16/308,990

(22) PCT Filed: Jul. 14, 2017
(86) PCT No.: PCT/IB2017/054281

§ 371 Date: Dec. 11, 2018

(30) Foreign Application Priority Data
Jul. 14, 2016 (PT) 109532

(51) Int. Cl. C12Q 1/6883

(52) U.S. Cl. CPC C12Q 1/6883; C12Q 2600/158

(57) ABSTRACT

A new molecular based method for early screening of twin-to-twin transfusion syndrome (TTTS) of monochorionic twin pregnancies is provided in a rapid and non-invasive manner. Using real time quantitative PCR, this method is intended to quantify the expression of HBB, a new TTTS biomarker, in maternal plasma. In pregnant women with this condition, this biomarker is significantly reduced when compared to non-pregnant women, as well as pregnant women with both single and twin pregnancies without pathology. This method, if applied to all women with monochorionic twin pregnancies, will allow early screening of TTTS, benefiting both the mother and the fetuses, which can more quickly take advantage of an effective treatment with predictable improvement in fetuses' survival.

### TECHNICAL DOMAIN

The present patent application refers to a new method of detection of twin-to-twin transfusion syndrome (TTTS) applicable to all monochorionic twin pregnancies.

### BACKGROUND ART

Twin pregnancies of monochorionic placentation have a relatively constant incidence (˜1 million pregnancies/year) and are not subject to the effects of heredity, ethnicity or assisted reproduction techniques as in dichorionic pregnancy. Pre- and perinatal morbidity and mortality in multiple pregnancies is clearly superior to unifetal pregnancy varying, however, according to chorionicity, many complications may occur for the mother and the foetus.

The twin-to-twin transfusion syndrome (TTTS) is a serious medical condition that occurs in 10 to 15% of twin pregnancies with monochorionic/diamniotic placentation. TTTS is associated to a high risk of fetal and perinatal mortality ranging from 60 to 100%, as well as occurrence of severe neurological sequelae in about 20 to 30% of the surviving fetuses. The diagnostic method consists of ultrasound evaluation in the 2nd and 3rd trimesters of pregnancy. However, the incidence of TTTS may be underestimated, as some foetal deaths in monochorionic pregnancies occur in early pregnancy and may be due to undiagnosed cases of TTTS. Although well characterized by ultrasonography, the aetiology of the syndrome remains poorly understood, and the occurrence of placental anastomoses between the twins seems to be the key to the pathophysiology of TTTS. The hemodynamic imbalance between fetuses is responsible for the hypoperfusion of the donor twin and for the hyperperfusion of the recipient twin. Thus, the clinical manifestations resulting include hypovolemia, growth restriction, high cardiac output and oligohydramnios in the donor twin, and fluid overload, congestive heart failure and polyhydramnios in the recipient twin. When TTTS occurs at an early gestational age it appears to have a worse prognosis than in later stages of the pregnancy. The laser ablation of the placental anastomoses, in order to convert a monochorionic placenta in a functional dichorionic placenta, is currently the most appropriate treatment for TTTS between 16 and 26 weeks of pregnancy, and without treatment the most likely outcome will be death of both twins. However, therapeutic interventions diminish but do not eliminate the risk of sequelae in the neuro-development of the surviving fetuses, since many times TTTS is diagnosed when many and serious complications are already installed. In short, a new method that enables the earlier identification of TTTS will represent an added value to its treatment and will lead to a decrease both in foetal mortality or the risk of sequelae in the surviving twins.

### SUMMARY

The present application relates to a method to detect the HBB gene for early identification of twin-to-twin transfusion syndrome in biologic samples comprising the following steps:
maternal blood collection at the time of the biochemical screening of the 1st quarter or at another time of pregnancy; obtaining RNA to test, by plasma separation and extraction of cell free RNA (cfRNA) from maternal blood;
transformation of cfRNA into cDNA; preparation of compositions for PCR comprising primers and probes with sequences SEQ ID nr.1 to SEQ ID nr.6, to which the cDNA to be tested is added;
analysis of the results based on the amplification curves and quantification of HBB and 18S (endogenous control) genes in the cDNA sample.

In one embodiment the method is performed up to the 16^{th} week of pregnancy.

In another embodiment the sample of RNA to test is obtained from amniotic liquid or plasma.

In an embodiment the results are obtained up to 48 hours after collecting the samples.

### GENERAL DESCRIPTION

The present application refers to a primer set and probe sequences (SEQ ID nr.1 to SEQ ID nr.6), for amplification and hybridization of DNA from biological samples, which were designed to present high specificity in detection of HBB transcripts.

Since these transcripts are present at lower levels in TTTS cases, these genetic probes and primers allow for the development of a highly sensitive method for TTTS detection.

The present application also refers to compositions for DNA amplification and hybridization, using the PCR technique (Polymerase Chain Reaction) comprising said sequences of primers and DNA probes, as well as others that are directed towards detection of HBB transcripts.

These compositions are applicable to TTTS detection and may be advantageously pre-prepared or being prepared at the time of the detection method in this invention.

Another aspect of this application relates to compositions for the detection of TTTS, comprising compositions for PCR plus human DNA from a biological sample to be tested for that purpose.

In these compositions, the amount of DNA in the test sample can be quite low, given the high specificity of the primers and probes of the present invention designed for that purpose.

Another aspect of the present application relates to a kit comprising the primer composition and specific genetic probes for the detection of TTTS, which can be, in this case, pre-prepared and part of said kit.

This kit has the advantage of developing a TTTS detection method, in a rapid and reliable manner, through the addition of a human DNA sample to test for the detection of TTTS syndrome.

Another aspect of the present application relates to a method of in vitro detection of TTTS, using reaction compositions comprising primers for amplification and probes whose sequences are specific to the HBB gene and of an endogenous control, according to the list of sequences.

This method may be advantageously used in TTTS detection by quantifying the transcripts of HBB present in biological samples conferred by the gene sequences designed for this purpose.

The major advantages of the method herein described, imperative for monochorionic twin pregnancies, are their rapid, until 48 hours after collecting the samples, non-invasive nature, as well as the capacity for early TTTS detection, a syndrome that involves a high risk of fetal loss, perinatal deaths and sequelae in survivors.

This method is fast, since it is based on the PCR technique in real time, and is non-invasive, as it is done in maternal plasma. Early detection arises, on one hand, from the comparison with the current method of diagnosis of TTTS, and on the other hand, from the biomarker efficiency on the tested groups. Currently, the detection of TTTS is made by sonographic evaluations from the 16th week of gestation onwards. The method described herein is based only on prior knowledge of the pregnancy chorionicity, determined at 11-14 weeks of gestation. Therefore, the identification of the disease can be performed at an earlier stage of pregnancy, representing an advance over the current TTTS diagnosis.

On the other hand, the method has the advantage that the greatest differences in HBB gene expression are detected in the group of pregnant women with early TTTS diagnosis (Group 1, 16-20 weeks of gestation) in comparison with pregnant women without pathology. The application of this early TTTS screening method will allow a differentiated prenatal monitoring at referral centers and will increase the early detection of the disease, decreasing the risk of loss of both fetuses or sequelae in survivor fetuses.

In fact, the need to introduce TTTS screening tests based on ultrasonographic evaluation or biomolecular markers is a subject that has been discussed in the scientific community.

The method of quantification of HBB expression in maternal plasma described herein has the advantage of responding to an urgent need for early TTTS diagnosis, applicable to all monochorionic twin pregnancies. This method also has the advantage of allowing detection of a greater number of cases in less advanced stages of the disease, leading to higher success rates for the survival of both fetuses and decreasing fetal mortality and perinatal morbidity.

### BRIEF DESCRIPTION OF DRAWINGS

For an easier understanding of the present patent application, some figures have been prepared and are attached to present examples, that do not intend to limit the herein communicated technology.

FIG. 1 shows HBB expression in plasma cfRNA from women with TTTS pregnancies and controls. A—women with TTTS pregnancies (n=18); B—non-pregnant women (n=10); C—women with unifetal pregnancies without pathology (n=10); D—women with twin pregnancies without pathology (n=14).

FIG. 2 shows HBB expression in plasma cfRNA from women with TTTS pregnancies and controls. A—Group 1 TTTS pregnancies 16-20 weeks of gestation (n=8); B—Group 2 TTTS pregnancies 20-24 weeks of gestation (n=5); C—Group 3 TTTS pregnancies over 25 weeks of gestation (n=5); D—non-pregnant women (n=10); E—women with unifetal pregnancies without pathology 15-24 gestation weeks (n=10); F—women 17-29 gestation weeks with twin pregnancies without pathology (n=14).

FIG. 3 shows p-values found for the comparisons between HBB expression in the plasma of TTTS pregnant women, non-pregnant women, women carrying unifetal pregnancies and women with twin pregnancies without pathology. In grey are statistically significant comparisons (ANOVA, p<5.00E-02).

### DETAILED DESCRIPTION

Several studies have shown that the amniotic fluid is a source of fetal free nucleic acids (cell free fetal DNA/RNA, cffDNA, cffRNA). In particular, the cffRNA can be isolated from the amniotic fluid for gene expression evaluation using large or small scale technologies, such as microarray or real-time PCR, respectively.

To better understand the aetiology of TTTS, cffRNA was isolated from 18 cases of receptor-fetuses with TTTS. The gene expression profiles of each of these samples was analysed using gene expression microarrays and compared with the profile obtained for 5-cffRNA control-cases from single pregnancies without pathology. After real-time PCR validation, the HBB gene was identified as differentially expressed in the amniotic fluid of the recipient fetuses. This gene is responsible for encoding the R subunit of hemoglobin, a metalloproteinase which, in its mature form, is a tetramer of two a subunits and two R subunits.

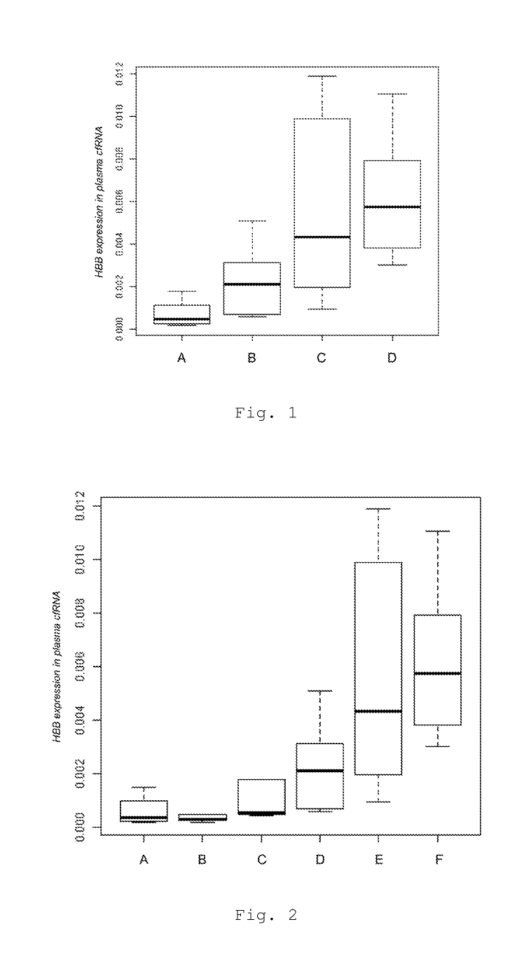

Next, expression of the biomarker was evaluated in maternal blood plasma, particularly in free nucleic acids (cell free RNA, cfRNA). Importantly, in maternal plasma, the fraction of cffRNA is considered small (<10%) when compared to the maternal fraction (>90%). Using real-time PCR, it was observed that the expression of HBB was significantly decreased in plasma cfRNA from mothers with TTTS syndrome compared with either plasma cfRNA from pregnant women with unifetal pregnancies (n=10) or pregnant women with twin pregnancies without disease (n=14) (FIG. 1, FIG. 3). Note that HBB expression in maternal plasma cfRNA was increased in the control group of women with unifetal pregnancies and even higher in the control group of women with twin pregnancies, relative to the female control group of non-pregnant women (n=10) (FIG. 1, FIG. 3). This may be explained by the fact that there is a greater production of hemoglobin from the first weeks of the pregnancy onwards, and that such production can be greater in gestation of higher parity.

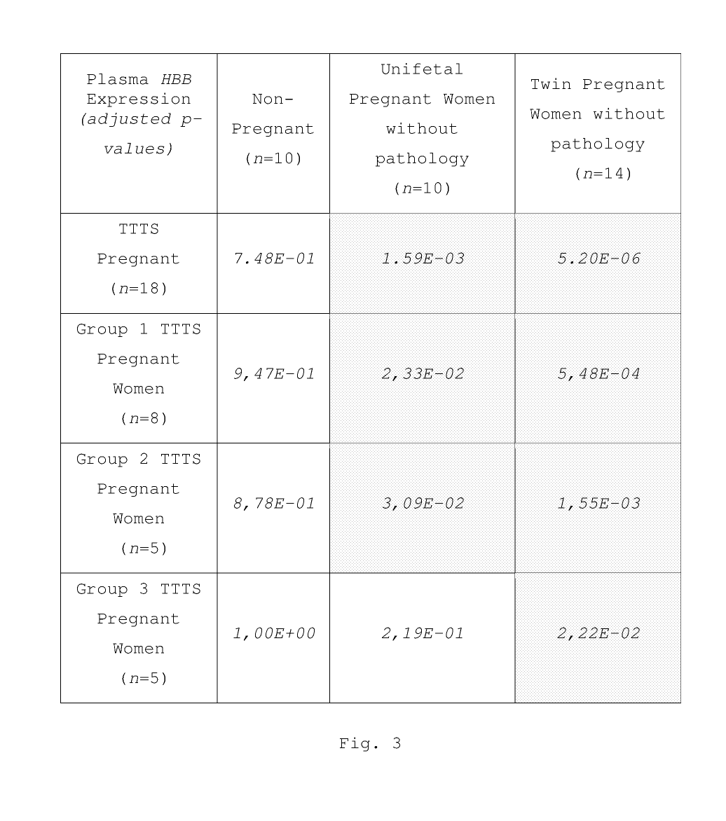

Next, samples were separated taking into account the gestation period at the time of plasma collection from pregnant women with pathology: group 1, 16-20 weeks of gestation; group 2, 20-24 weeks of gestation and group 3, over 25 weeks of gestation. It was observed that for group 1, the expression values of HBB were recorded as the lowest among the 3 groups (FIG. 2, FIG. 3). Together, this information supports that HBB is a new TTTS biomarker, with particular emphasis on early detection of the disease.

The present patent application refers to a method for detection of twin-to-twin transfusion syndrome (TTTS). This method uses reaction compositions comprising nucleotide sequences of primers for amplification and probes for detection, all of which unique and specific for HBB gene (SEQ ID nr.1 SEQ ID nr.6).

Thus, primer sequences were created for amplification and probes for detection in order to build the basis for the amplification reaction, eg. PCR assay a real time (RT-PCR). Six primers and probes sequences were designed, as shown in the sequence list.

The indicated sequences can be prepared by the method of nucleotide synthesis in solid phase using phosphoramidite nucleosides as described in the publication: Beaucage, S. L.; Iyer, R. P. (1992). “Advances in the Synthesis of Oligonucleotides by the Phosphoramidite Approach”.

Tetrahedron 48 (12): 2223.

These can be further modified at the 5′ and 3′ sequences of the probes by addition of fluorophores, such as FAM, Yakima Yellow, quencher (TAMRA) for the purpose of amplification reactions and hybridization in Multiplex PCR. In one embodiment, the suitable fluorophores in the scope of this present patent application are compounds known as Alexa Fluor FAM, TET, JOE, VIC, HEX, Cy3, ATTO 550, TAMRA, ROX, Cy5, Cy5.5.

The nucleotides refer to nucleotides of natural or synthetic origin, with the hybridization capacity by base-pairing with complementary nucleotides, and may include, without limitation, DNA, RNA, and nucleotide analogues (e.g. nucleic acids with closed conformation, known as “locked nucleic acids”—LNA) or nucleotides without inter-nucleotide phosphodiester linkages (e.g. peptide nucleic acid—PNA) or nucleic acids with thioester bonds, or other similar for the same purpose.

Compositions for amplification and DNA hybridization (PCR compositions) were prepared by adding reaction solutions to PCR, available on the market, with different nucleotide sequences with sequences SEQ ID Nr.1 to SEQ ID nr.6 in different concentrations, and bi-distilled and bi-deionized water.

The reaction solutions for PCR to be used vary with the desired type of amplification reaction. Examples of suitable reaction solutions for PCR for the embodiment of the present application are for example the solution “TaqMan® Universal PCR Master Mix” from the company Thermofisher Scientific or similar solutions, for the purpose of DNA amplification and hybridization.

The concentrations of probes refer to the molar concentration of the solution, corresponding to the number of moles of each probe per volume of solution, wherein nanoMolar (nM) corresponds to 1×10⁻⁹ moles per litre.

In one embodiment, the method herein described comprises 5 steps:
Maternal blood collection at the time of the biochemical screening of the 1st quarter or at another time of pregnancy;
Obtaining RNA to test, by plasma separation and extraction of cell free RNA (cfRNA) from maternal blood using a commercial kit or similar methods. In an embodiment, the comercial kit is QIAamp Circulating Nucleic Acid Kit QIAGEN®;
Transformation of cfRNA into cDNA using a commercial kit or similar methods. In one embodiment the comercial kit is SuperScript® VILO cDNA Synthesis Kit ThermoScientific®;
Preparation of compositions for PCR comprising primers and probes with sequences SEQ ID nr.1 to SEQ ID nr.6, to which the cDNA to be tested is added.
Analysis of the results based on the amplification curves and quantification of HBB and 18S (endogenous control) genes in the cDNA sample.

In another embodiment, the method is performed up to the 16^{th} week of pregnancy.

The present description is, naturally, not in any way restricted to the embodiments presented herein and a person of ordinary skill in the art may foresee many possibilities of modifying it without departing from the general idea as defined in the claims. The preferred embodiments described above are obviously combinable with each other. The following claims further define preferred embodiments.

### SEQUENCE LIST

SEQ ID 1: GCTGTCCAATTTCTATTAAAGGTTCC
SEQ ID 2: GGCAGAATCCAGATGCTCAA
SEQ ID 3: GAGACTCTGGCATGCTAACTAG
SEQ ID 4: GGACATCTAAGGGCATCACAG
SEQ ID 5: TGTTCCCTAAGTCCAACTACTAAACTGGG
SEQ ID 6: TGCTCAATCTCGGGTGGCTGAA

1. Method to detect HBB gene expression for early identification of twin-to-twin transfusion syndrome biologic samples comprising:
obtaining RNA to test, by separation and extraction of cell free RNA (cfRNA) from maternal plasma or amniotic fluid;
transforming cfRNA into cDNA: preparation of compositions for PCR comprising primers and probes with sequences SEQ ID nr.1 to SEQ ID nr.6, to which the cDNA to be tested is added;
analyzing results based on amplification curves and quantification of HBB and 18S endogenous control genes in the cDNA sample.

2. Method to detect the HBB gene expression according to claim 1, wherein the method is performed up to the 16th week of pregnancy.

3. Method to detect the HBB gene expression according to claim 1, wherein the results are obtained up to 48 hours after collecting the samples.
